(19) United States
(12) Patent Application Publication
(10) Pub. No.: US 2016/0353975 A1
(43) Pub. Date: Dec. 8, 2016

(54) BENDING OPERATION MECHANISM FOR ENDOSCOPE

(71) Applicant: OLYMPUS CORPORATION, Tokyo (JP)

(72) Inventor: Masanori HAMAZAKI, Tokyo (JP)

(73) Assignee: OLYMPUS CORPORATION, Tokyo (JP)

(21) Appl. No.: 15/244,443

(22) Filed: Aug. 23, 2016

Related U.S. Application Data
(63) Continuation of application No. PCT/JP2015/057457, filed on Mar. 13, 2015.

(30) Foreign Application Priority Data
Jun. 24, 2014 (JP) 2014-129639

(51) Int. Cl. A61B 1/005

(52) U.S. Cl. CPC A61B 1/0053; A61B 1/0057

(57) ABSTRACT

A bending operation mechanism for endoscope according to the invention includes: a bending operation member to which a bending wire for driving a bending portion of an endoscope, the bending operation member being rotationally operated to pull the bending wire in a rotation direction; a rotator coupled to the bending operation member and configured to be rotationally driven by rotation operation of the bending operation member; a reduction gear mechanism that reduces rotation of the rotator; and a biasing mechanism that applies a biasing force in a rotation direction to an output gear in the reduction gear mechanism.

### CROSS REFERENCE TO RELATED APPLICATION

This application is a continuation application of PCT/JP2015/057457 filed on Mar. 13, 2015 and claims benefit of Japanese Application No. 2014-129639 filed in Japan on Jun. 24, 2014, the entire contents of which are incorporated herein by this reference.

### BACKGROUND OF THE INVENTION

1. Field of the Invention

The present invention relates to a bending operation mechanism for an endoscope which includes a bending portion on a distal end side of an insertion portion and performs bending operation of the bending portion with a bending operation member provided at an operation portion located on the proximal end side with respect to the insertion portion.

2. Description of the Related Art

In recent years, endoscopes have been widely used in medical fields and industrial fields. The endoscopes of such types include an endoscope having an elongated flexible insertion portion and a bending portion provided on the distal end side of the insertion portion. The bending portion is configured to be able to be bent in a desired direction by operating a bending operation member provided at the operation portion located on the proximal end side with respect to the insertion portion. The bending portion is thus bent, to thereby change the observation direction of the observation optical system at the distal end portion arranged on the more distal end side with respect to the bending portion, to enable an examination to be performed in a wide range.

In conventional endoscopes, the bending operation member provided at the operation portion includes a joystick type lever, or the like, which is configured to be tilted, as well as a bending knob, a bending lever, or the like, which is configured to be rotated around an axis.

Furthermore, as the bending operation mechanism provided with the bending operation member of such a type, the bending operation mechanism has a configuration as described below has generally been put to practical use. The bending operation mechanism includes, for example: a bending portion provided on a distal end side of an insertion portion and formed by coupling a plurality of joint rings with one another; a bending operation member disposed at an operation portion provided on a proximal end side with respect to the insertion portion; a rotator such as a sprocket or a pulley which is provided in the operation portion and configured to be rotatable integrally with the bending operation member in accordance with the rotation operation of the bending operation member; and a plurality of angle wires, each of which has one end fixed to the bending portion and the other end fixed to the rotator and is inserted in the insertion portion, and the bending operation mechanism is configured to pull the angle wires by rotating the bending operation member to cause the rotator to rotate.

In such a conventional endoscope having a common configuration, the bending portion provided at the insertion portion is covered with a bending rubber having elasticity. Inside the insertion portion and the bending portion of such an endoscope, a plurality of endoscope internal components, for example, a treatment instrument channel tube, an air feeding tube, a water feeding tube, a signal cable formed by bundling a plurality of signal lines, an illumination light guide, and the like are inserted and placed.

The bending rubber and the endoscope internal components cause elastic resistance when the bending portion is bent. That is, when the bending portion is bent, an elastic restoring force for trying to return the bending portion to a linear state acts due to the bending rubber and the endoscope internal components. At the same time, a frictional force is generated by contact among the internal components when the bending portion is bent.

FIG. 6, for example, shows a relationship between an amount of bending operation force used at the time when the bending operation member is operated and a bending angle of a bending portion in an endoscope. In FIG. 6, the dotted lines shown by the reference numeral L1 represents the elastic restoring force (conventional) for trying to return the bending portion to the linear state by the bending rubber and the endoscope internal components in a conventional endoscope. In addition, in FIG. 6, the dotted lines shown by the reference numeral L2 represents a resistance force (conventional) such as the frictional force generated by the contact among the internal components when the bending portion is bent in the conventional endoscope. The amount of force as a result of adding the elastic restoring force (conventional) L1 and the resistance force (conventional) L2 tends to increase as the bending angle of the bending portion increases, as shown in FIG. 6.

Therefore, when the user performs operation for bending the bending portion, the user needs to operate the bending operation member with an amount of force against the resistance force. As the bending angle of the bending portion increases, the resistance force increases. That is, in order to bend the bending portion against the resistance force, the amount of bending operation force for operating the bending operation member is also required to be increased. Such a circumstance is possibly pointed out as a problem of increase in the load to be applied to the user's hand and fingers with which the operator performs the bending operation.

In view of the above, for example, Japanese Patent Application Laid-Open Publication No. 2012-81012 discloses various kinds of configurations of the bending operation member for endoscope that is capable of reducing an operation force of angle wire pulling means.

### SUMMARY OF THE INVENTION

A bending operation mechanism for endoscope according to one aspect of the present invention includes: a bending operation member to which a bending wire for driving a bending portion of an endoscope is coupled, the bending operation member being rotationally operated to pull the bending wire in a rotation direction; a rotator coupled to the bending operation member and configured to be rotationally driven by rotation operation of the bending operation member; a reduction gear mechanism that reduces rotation of the rotator; and a biasing mechanism that applies a biasing force in a rotation direction to an output gear in the reduction gear mechanism.

Furthermore, a bending operation mechanism for endoscope according to another aspect of the present invention includes: a bending operation member to which a bending wire for driving a bending portion of an endoscope is coupled, the bending operation member being rotationally operated to pull the bending wire in a rotation direction; a first gear coupled to the bending operation member and rotationally driven by rotation operation of the bending operation member; a reduction gear mechanism coupled to the first gear and including a second gear to which rotation of the first gear is transmitted after the rotation is reduced; and a biasing mechanism that applies a biasing force in a rotation direction to the second gear, wherein the biasing mechanism includes a proximal end rotatably and pivotally supported at an immovable portion and a distal end rotatably and pivotally supported at an outer diameter portion of the second gear, and when the bending operation member is in a neutral state in which the bending portion is not bent and in substantially a linear state, two axes of the biasing mechanism and a rotational axis of the second gear are arranged so as to align on substantially a straight line.

### BRIEF DESCRIPTION OF THE DRAWINGS

FIG. 1 is a schematic configuration diagram showing an overall configuration of an endoscope to which a bending operation mechanism according to an embodiment of the present invention is applied.

FIG. 2 is a main-part enlarged cross-sectional view showing a part of an internal configuration of an operation portion in the endoscope in FIG. 1.

FIG. 3 is a basic configuration diagram of a bending operation assisting mechanism unit applied to the bending operation mechanism in the endoscope in FIG. 1 (in the case where the bending operation member is in the neutral state).

FIG. 4 illustrates the bending operation assisting mechanism unit in a case where rotation operation of the bending operation member is performed to bring the bending portion into a maximum bending state in the bending operation assisting mechanism unit in the state shown in FIG. 3.

FIG. 5 illustrates the bending operation assisting mechanism unit in a case where the rotation operation of the bending operation member is performed and the bending portion is in the middle of being bent in the bending operation assisting mechanism unit in the state shown in FIG. 3.

FIG. 6 illustrates a relationship between the amount of bending operation force at the time when the bending operation member is operated and a bending angle of a bending portion in a conventional endoscope.

### DETAILED DESCRIPTION OF THE PREFERRED EMBODIMENT(S)

Hereinafter, description will be made on the present invention with reference to the embodiment shown in the drawings. In the drawings used in the description below, there is a case where a different scale size is used for each of the constituent elements in order to allow each of the constituent elements to be illustrated in a recognizable size in the drawings. Therefore, the present invention is not limited only to the number and shapes of the constituent elements, a ratio of the size of one constituent element to that of another constituent element, and a relative positional relationship among the constituent elements shown in these drawings.

First, brief description will be made below on an overall configuration of an endoscope including a bending operation mechanism according to an embodiment of the present invention and a schematic configuration of the bending operation mechanism for the endoscope.

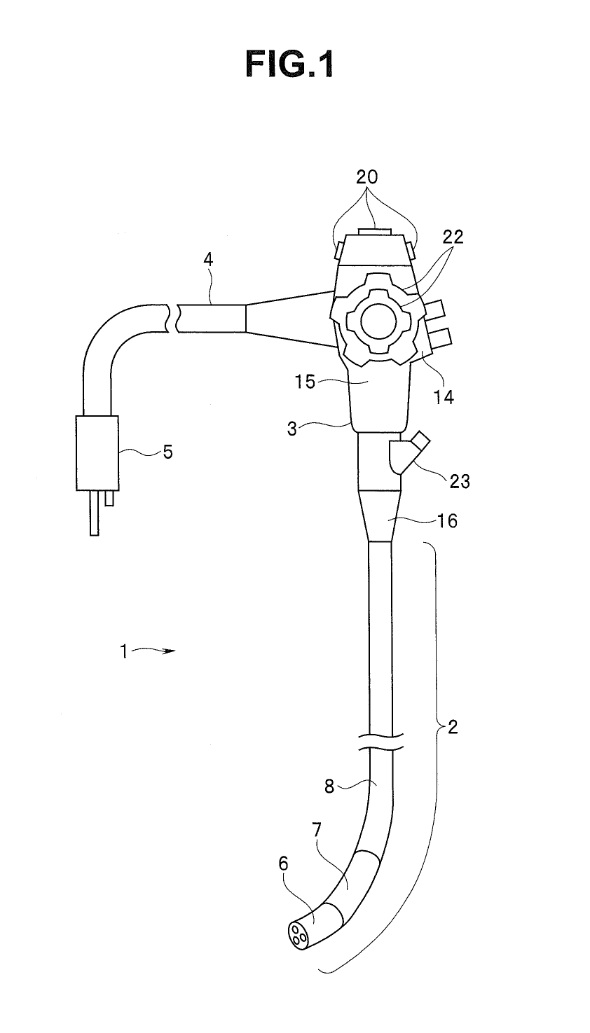

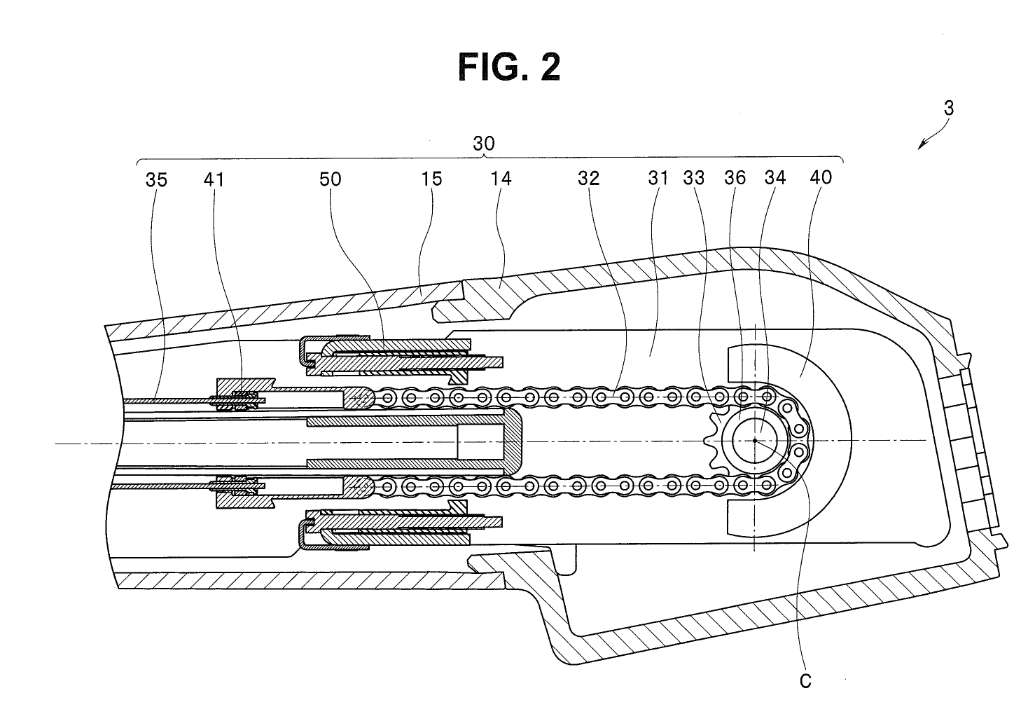

FIG. 1 is a schematic configuration diagram showing the overall configuration of the endoscope to which the bending operation mechanism according to the embodiment of the present invention is applied. FIG. 2 is a main-part enlarged cross-sectional view showing a part of an internal configuration of an operation portion in the endoscope in FIG. 1.

An endoscope 1 including a bending operation mechanism 30 according to the present embodiment is mainly configured by: an elongated insertion portion 2 configured to be inserted into a body cavity; an operation portion 3 provided continuously with the proximal end side of the insertion portion 2; a universal cord 4 whose proximal end portion is coupled to the one side surface of the operation portion 3; a connector 5 disposed at the distal end portion of the universal cord 4; a bending operation mechanism 30; a bending angle adjusting mechanism 50 (not shown in FIG. 1, see FIG. 2) included in the bending operation mechanism 30; and the like. Note that the endoscope 1 is configured to operate as an endoscope system by being connected to a light source apparatus and a control apparatus such as a video processor, which are not shown, through the connector 5.

The insertion portion 2 is configured by coupling a rigid distal end constituting portion 6, a bending portion 7, an elongated flexible tube portion 8 having flexibility in this order from the distal end. The bending portion 7 is configured to be able to be bent in four directions, that is, up and down directions, and left and right directions by the bending operation mechanism 30 (see FIG. 2), and the bending portion can be bent in an arbitrary direction by combining the bending operations in the four directions.

On the distal end surface of the distal end constituting portion 6, an objective lens, an illumination lens, a cleaning nozzle, a treatment instrument channel opening, and the like are disposed (not shown). In addition, inside the distal end constituting portion 6, not only an image pickup device, electric parts such as an electric substrate, and a video cable extended from the image pickup device, but also an air feeding conduit, a water feeding conduit and the like coupled to the cleaning nozzle, a light guide fiber that supplies illumination light to the illumination lens, etc. (not shown), are disposed. The video cable and the light guide fiber are inserted through the insertion portion 2, the operation portion 3 and the universal cord 4, to be extended to the connector 5. In addition, the air feeding conduit and the water feeding conduit are inserted through the insertion portion 2 to be extended to the connector 5 through an air/water feeding cylinder provided in the operation portion 3 and the universal cord 4. Note that the outer surface and the internal configuration of the distal end constituting portion 6 are supposed to be the same as those in a conventional common type of endoscope, and detailed description and illustration thereof will be omitted.

The operation portion 3 is formed watertightly by housing members such as an external housing 14, a grasping portion housing 15, and the like. The proximal end portion of the insertion portion 2 is coupled to the one end portion of the grasping portion housing 15. At the coupling part between the proximal end portion of the insertion portion 2 and the one end portion of the grasping portion housing 15, for example, a bend prevention portion 16 made of an elastic rubber member or the like, for example, is provided in order to prevent the flexible tube portion 8 from being abruptly bent.

On the external housing 14 of the operation portion 3, a plurality of bending operation members 22 for performing bending operation of the bending portion 7 of the insertion portion 2 are rotatably disposed so as to be coaxial with a spindle 34 (see FIG. 2) made of an axis member. The plurality of bending operation members 22 are mechanically connected to the bending operation mechanism 30 (see FIG. 2) disposed in the operation portion 3, that is, in the external housing 14 and the grasping portion housing 15.

In addition, on the outer surface of the external housing 14 of the operation portion 3, various kinds of operation members, for example, a plurality of switches 20 for remotely operating peripheral devices (not shown) such as a video processor and the like are provided. Furthermore, on the outer surface of the grasping portion housing 15, a treatment instrument introducing port 23 through which a treatment instrument and the like, not shown, are introduced is provided. The treatment instrument introducing port 23 communicates with a treatment instrument channel (not shown) inside. The treatment instrument channel is inserted through the insertion portion 2 to reach the treatment instrument channel opening located at the distal end constituting portion 6.

As shown in FIG. 2, inside the operation portion 3, a main frame 31 which is a skeleton member and which fixes and supports various kinds of constituent members, and a plurality of configuration units such as the bending operation mechanism 30 are disposed.

The main frame 31 is a structure formed in a ladder structure or a plate structure, for example, by die-casting for injection-molding a metal member such as aluminum. The main frame 31 is fixed on the inner surfaces of the external housing 14 and the grasping portion housing 15 with screws, for example, in the operation portion 3. The bending operation mechanism 30 is held by being fixed to the main frame 31, which is disposed inside the operation portion 3, through the use of screws or the like.

The bending operation mechanism 30 is a mechanism unit for enabling the bending motion of the bending portion 7. When the user rotates the bending operation member 22 (not shown in FIG. 2, see FIG. 1) while grasping the operation portion 3, the bending operation wires (bending operation wires 35) whose end portions are fixed to the bending portion 7 on the distal end side of the insertion portion 2 and which are configured to drive the bending portion 7, move back and forth in the direction along the longitudinal axis of the insertion portion 2, to thereby enable the bending portion 7 to be bent. To this end, the bending operation mechanism 30 is configured as a pair of configuration units formed by combining a left/right bending operation mechanism that enables the bending portion 7 to be bent in left and right directions and an up/down bending operation mechanism that enables the bending portion 7 to be bent in up and down directions. The left/right bending operation mechanism and the up/down bending operation mechanism have substantially the same configuration, and disposed in the operation portion 3 in a layered manner in the axial direction of the spindle 34 to be described later. Therefore, in the present embodiment, in order to avoid complication of the drawings, only one of the left/right bending operation mechanism and the up/down bending operation mechanism that configure the bending operation mechanism 30 is shown to simplify the drawings, and detailed description will be made on only one of the left/right bending operation mechanism and the up/down bending operation mechanism.

The bending operation mechanism 30 includes the bending operation member 22, and configured by the spindle 34, a sprocket 33 (rotator), a chain 32, a cover member 40, the bending angle adjusting mechanism 50, and the bending operation wires 35 (bending wires) and the like.

The spindle 34 is a rod-shaped member, the lower end of which is embedded and fixed to the main frame 31. The upper end of the spindle 34 protrudes outside the external housing 14. A cylindrical body 36 is rotatably arranged on the outer circumferential side of the spindle 34. In other words, the spindle 34 pivotally supports the cylindrical body 36 such that the cylindrical body 36 is rotatable. The bending operation member 22 is fixedly provided at the upper end of the cylindrical body 36. In addition, the sprocket 33 is fixedly provided at the lower end of the cylindrical body 36. According to such a configuration, the sprocket 33 is mechanically coupled to the bending operation member 22 through the cylindrical body 36.

A chain 32 is wound around the outer circumference of the sprocket 33 to be meshed with the sprocket. The bending operation wires 35 are joined respectively to the both ends of the chain 32 through coupling members 41.

In detail, the coupling members 41 are fixedly provided respectively at the both end portions of the chain 32. The proximal end portion of each of the bending operation wires 35 inserted in the insertion portion 2 is provided so as to be continuous with each of the coupling members 41. The distal end portion of each of the bending operation wires 35 is fixed to the distal end part (not shown) of the bending portion 7 of the insertion portion 2. Each of the bending operation wires 35 is formed by twisting a plurality of wires, and stranded wires having flexibility are used as the bending operation wires.

In the vicinity of the coupling part between the chain 32 and the bending operation wires 35, the bending angle adjusting mechanism 50 is disposed. The bending angle adjusting mechanism 50 is a mechanism unit for setting the respective maximum bending angles of the bending portion 7 in the four directions, that is, the up and down directions and the left and right directions. The bending angle adjusting mechanism 50 is fixed to the main frame 31 with screws, for example. Note that, since the bending angle adjusting mechanism 50 is a part not directly related to the configuration of the present embodiment, the bending angle adjusting mechanism same as that applied to a conventional common endoscope is supposed to be applied to the present embodiment, and detailed description thereof will be omitted.

In addition, the cover member 40 is a member provided so as to cover the vicinity of the outer circumferential side of the sprocket 33 to prevent the chain 32 wound around the outer circumference of the sprocket 33 from falling off. The cover member 40 is fixed to the main frame 31 with screws, for example.

Note that, in FIG. 2, the reference numeral C represents rotation center axes of the spindle 34, sprocket 33, and cylindrical body 36. That is, in the present embodiment, the rotation center axis C also serves as the rotation center axis of the bending operation member 22 (not shown in FIG. 2).

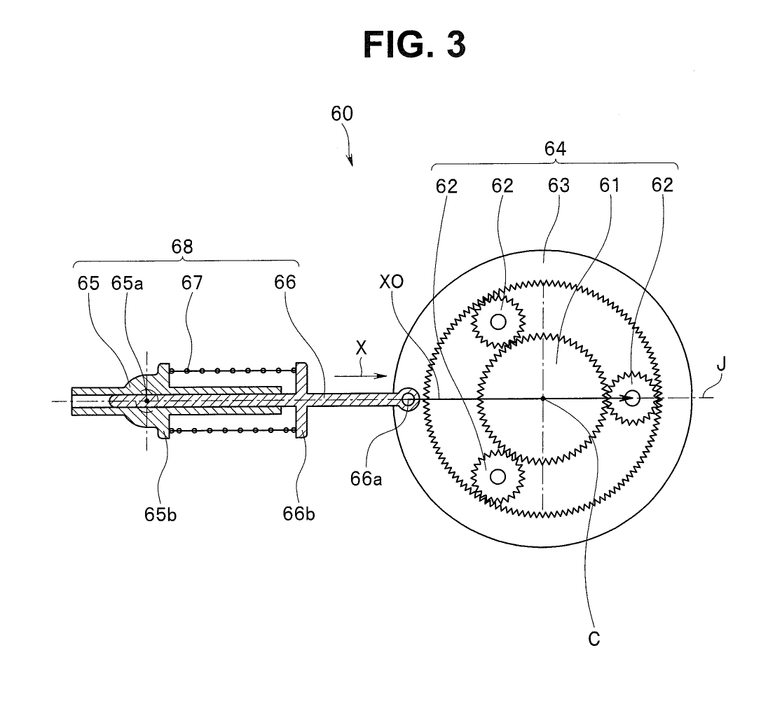

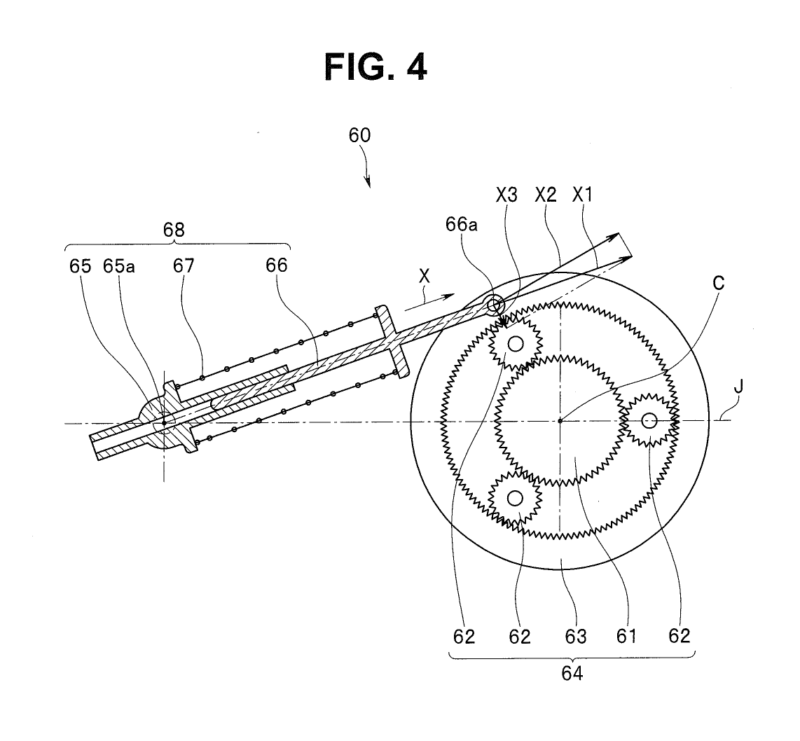

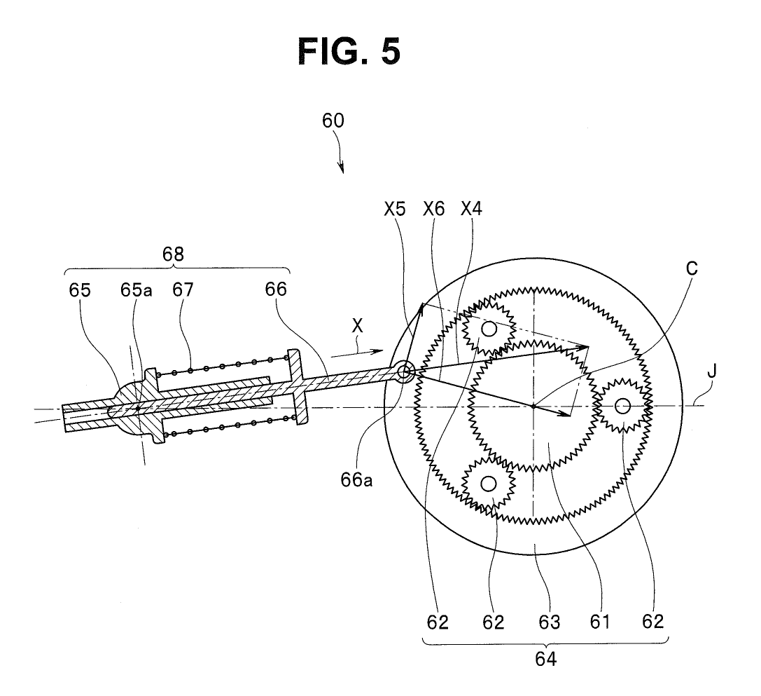

In addition, the bending operation mechanism 30 according to the embodiment of the present invention includes a bending operation assisting mechanism unit 60 for assisting the bending operation performed with the bending operation member 22, in addition to the above-described configuration. Description will be made below on the configuration of the bending operation assisting mechanism unit 60, with reference to FIGS. 3 to 6. FIGS. 3 to 5 are basic configuration diagrams of the bending operation assisting mechanism unit applied to the bending operation mechanism in the present embodiment. Among FIGS. 3 to 5, FIG. 3 illustrates the state of the bending operation assisting mechanism unit in the case where the bending operation member is in the neutral state. FIG. 4 illustrates the state of the bending operation assisting mechanism unit in a case where rotation operation of the bending operation member is performed to bring the bending portion into the maximum bending state. FIG. 5 illustrates the state of the bending operation assisting mechanism unit in a case where the rotation operation of the bending operation member is performed and the bending portion is in the middle of being bent.

The bending operation assisting mechanism unit 60 is configured by a planetary gear mechanism 64, a biasing mechanism 68, and the like. The planetary gear mechanism 64 is a reduction gear mechanism for reducing rotation operation amount of the bending operation member 22 (and the rotation of the rotator (sprocket 33), to be described later, configured integrally with the bending operation member 22). The biasing mechanism 68 is a biasing mechanism unit that reduces the amount of the rotation operation force of the bending operation member 22 by applying a biasing force in the rotation direction to an output gear (internal gear 63), to be described later, in the planetary gear mechanism 64 (reduction gear mechanism).

The planetary gear mechanism 64 is a planetary gear mechanism configured by a sun gear 61, planetary gears 62, the internal gear 63 (output gear), and the like. The sun gear 61 is disposed so as to be coaxial with the sprocket 33 (rotator) and configured to rotate integrally with the sprocket 33. As described above, the sprocket 33 in the present embodiment is a rotator to which the bending operation wires 35 are coupled through the chain 32 and which is configured integrally with the bending operation member 22 through the cylindrical body 36. That is, the sun gear 61 is configured to be rotatable integrally with the bending operation member 22, the cylindrical body 36, and the sprocket 33 in the same direction.

The sun gear 61 includes the plurality of planetary gears 62, which are a part of the configuration portion of the planetary gear mechanism 64, provided at equal intervals, and the teeth provided on the respective plurality of planetary gears are meshed with the teeth provided on the outer circumference of the sun gear 61. Each of the planetary gears 62 is rotatably and pivotally supported with respect to the fixed member inside the operation portion 3 of the endoscope 1, for example, the immovable portion such as the main frame 31. Note that the present embodiment shows an example in which the three planet gears 62 are provided. In addition, on the outer circumferential side of the plurality of planetary gears 62, the teeth of the above-described respective planetary gears 62 are meshed with the teeth provided on the inner circumference of the internal gear 63, which is a part of the configuration portion of the planetary gear mechanism 64 and which is the output gear.

According to such a configuration, when the bending operation member 22 is rotationally operated, the sprocket 33 rotates in the same direction through the cylindrical body 36. Simultaneously with such rotation, the sun gear of the planetary gear mechanism 64 rotates in the same direction. When the sun gear rotates in the planetary gear mechanism 64, in response to this rotation, the planetary gears 62 rotate in the direction opposite to the rotation direction of the sun gear, to thereby cause the internal gear 63 to rotate in the direction opposite to the rotation direction of the planetary gears. If it is supposed that the number of teeth of the sun gear 61 is Z1, the number of the teeth of the planetary gears 62 is Z2, and the number of the teeth of the internal gear 63 is Z3 in the planetary gear mechanism 64, the reduction gear ratio is calculated by the following equation, that is, the reduction gear ratio i=1+(Z3/Z1).

The biasing mechanism 68 is configured by a shaft 66, a cylinder 65, a compression spring 67, and the like.

The shaft 66 is formed by a rod-shaped member and includes a pin 66a at one end thereof. The pin 66a is coupled to one part of the internal gear 63 (outer diameter portion of the output gear) in the planetary gear mechanism 64. In the configuration, the pin 66a of the shaft 66 is coupled to the internal gear 63 so as to be rotatable at the coupling part between the shaft 66 and the internal gear 63. According to the configuration, the pin 66a of the shaft 66 serves as a pivot of the shaft 66. In addition, at the middle part of the shaft 66, a collar-like flange portion 66b is formed so as to protrude in the direction perpendicular to the longitudinal axis direction. The flange portion 66b is a part that functions as a spring receiving portion that receives one end of the compression spring 67 to be described later.

The cylinder 65 is formed by a hollow cylindrical member for pivotally support the shaft 66 such that the shaft 66 is slidable in the axis direction. Therefore, the other end side of the shaft 66 is arranged so as to be fitted in the hollow portion of the cylinder 65. In addition, the cylinder 65 is pivotally supported with respect to the fixed member inside the operation portion 3 of the endoscope 1, for example, the immovable portion such as the main frame 31, so as to be rotatable around the rotation center axis shown by the reference numeral 65a in FIG. 3, which is located at the proximal end of the cylinder 65. According to such a configuration, the rotation center axis 65a of the cylinder 65 is a pivot of the cylinder 65. Furthermore, at the middle part of the cylinder 65, a collar-like flange portion 65b is formed so as to protrude in the direction perpendicular to the longitudinal axis direction, similarly as in the above-described shaft 66. The flange portion 65b is a part that functions as a spring receiving portion that receives the other end of the compression spring 67 to be described later.

The compression spring 67 is an urging member made of a coil spring or the like, for example. The compression spring 67 is bridged between the flange portion 66b of the shaft 66 and the flange portion 65b of the cylinder 65, with energy being stored in the compression spring 67. According to such a configuration, the compression spring 67 biases the shaft 66 so as to push the shaft in the longitudinal axis direction with respect to the cylinder 65 which is in the fixed state.

The bending operation by using the endoscope 1 to which the bending operation mechanism 30 for endoscope configured as described above is applied is performed as described below. That is, the user rotationally operates the bending operation member 22 (actually, there are two bending operation members, that is, the bending operation member for up/down bending and the bending operation member for left/right bending). The amount of the rotational force of the bending operation member 22 is transmitted to the sprocket 33 through the cylindrical body 36, to cause the sprocket 33 to rotate in the direction same as the rotation direction of the bending operation member 22. When the sprocket 33 is thus rotated, the chain 32 is driven to travel in accordance with the rotation. Then, in accordance with the traveling of the chain 32, the bending operation wire 35 connected to the chain 32 through the coupling member 41 is driven to be pulled. This causes the bending portion 7 to be bent in the direction intended by the user, that is, one of the up and down directions or one of left and right directions by the amount corresponding to the rotation operation amount of the bending operation member 22.

In this case, when the bending operation member 22 is in the neutral state in which the bending operation member 22 is not rotationally operated, the bending operation assisting mechanism unit 60 is in the state as shown in FIG. 3. That is, as shown in FIG. 3, the shaft 66 fitted in the cylinder 65 is arranged such that the two axes of the biasing mechanism 68, that is, the rotation center axis 65a of the cylinder 65 and the pin 66a of the shaft 66 and the rotation center axis C of the sun gear 61 and the internal gear 63 (output gear) are arranged so as to align on a substantially straight line (along the linear line J in FIG. 3).

At this time, the compression spring 67 is in the energy storing state. Therefore, the shaft 66 is biased in the direction along the arrow X in FIG. 3. In this state, the biasing force of the compression spring 67 acts on the pin 66a of the shaft 66. That is, the entirety of the biasing force of the compression spring 67 works as an amount of force for pressing the pin 66a toward the center (rotation center axis C) of the planetary gear mechanism 64 (see the arrow XO). That is, the amount of the biasing force of the compression spring 67 which is applied in the direction of rotating the internal gear 63 through the shaft 66 is zero.

It is supposed that the rotation operation of the bending operation member 22 is performed in this state, to bring the bending portion into the maximum bending state, for example. In this case, when the bending operation member 22 is rotationally operated by a rotation angle of approximately 180 degrees, for example, the maximum bending angle of the bending portion 7 can be obtained. In addition, it is supposed that the reduction gear ratio i of the planetary gear mechanism 64 is 3, for example. In this case, when the bending operation member 22 is rotationally operated by the rotation angle of approximately 180 degrees, the sprocket 33 and the sun gear 61 rotate by the rotation amount same as that of the bending operation member, and the internal gear 63 rotates by a rotation angle of approximately 60 degrees. The state of the bending operation assisting mechanism unit 60 at this time is shown in FIG. 4.

As shown in FIG. 4, when the internal gear 63 rotates by the rotation angle of approximately 60 degrees in a predetermined direction (clockwise direction in FIG. 4) from the state shown in FIG. 3, the pin 66a of the shaft 66 moves in the direction (the clockwise direction in FIG. 4) same as the rotation direction of the internal gear 63, along the circular arc having the rotation center axis C as the rotation center. Then, the shaft 66 and the cylinder 65 rotate in the predetermined direction (counterclockwise direction in FIG. 4) by a predetermined amount with the rotation center axis 65a as the pivot. At this time, the shaft 66 is biased in the direction along the arrow X in FIG. 4 by the biasing force of the compression spring 67. In this state, the amount of force X1 with which the compression spring 67 presses the pin 66a of the shaft 66 is resolved into the amount of force X2 that acts in the direction in which the compression spring 67 causes the internal gear 63 to rotate and the amount of force X3 with which the compression spring 67 presses the planetary gear mechanism 64 toward the center thereof. At this time, the ratio of the amount of force X2 that acts in the direction of rotating the internal gear 63 is increased.

In this state, as described above, the bending operation for bringing the bending portion into the maximum bending state is performed with the bending operation member 22. The amount of bending operation force applied with the bending operation member 22 in the common endoscope 1 tends to be the largest when the bending portion is in the maximum bending state. In the endoscope 1 according to the present embodiment, when the bending portion is in the maximum bending state, the amount of force X2 with which the biasing force of the compression spring 67 presses the internal gear 63 in the direction of rotating the internal gear works on the internal gear 63. That is, the amount of pressing force X2 acts in the direction in which the bending portion is bent by the bending operation member 22. This reduces the amount of operation force required when the user performs bending operation with the bending operation member 22.

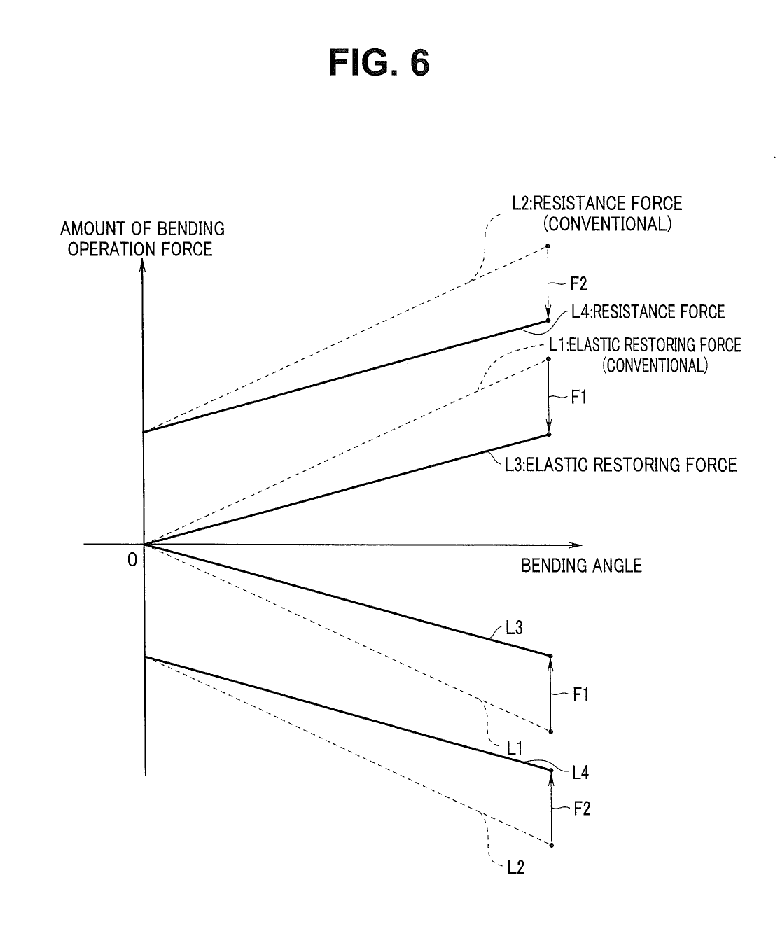

For example, the relationship between the amount of bending operation force when the user operates the bending operation member and the bending angle of the bending portion in the endoscope is as shown in FIG. 6. The dotted line shown with the reference numeral L1 in FIG. 6 represents the elastic restoring force (conventional) for trying to return the bending portion to the linear state by the bending rubber and the endoscope internal components in the conventional endoscope. In addition, the dotted line shown with the reference numeral L2 in FIG. 6 represents a resistance force (conventional) such as the frictional force generated by the contact among the internal components when the bending portion is bent in the conventional endoscope.

In view of the above, the endoscope 1 according to the present embodiment is provided with the bending operation assisting mechanism unit 60, to thereby reduce the amount of operation force when the user performs bending operation with the bending operation member 22 through the use of the biasing force of the compression spring 67. The solid lines shown with the reference numerals L3, L4 in FIG. 6 illustrate the above-described reduction of the amount of operation force. That is, the solid line shown with the reference numeral L3 in FIG. 6 represents the elastic restoring force of the endoscope according to the present embodiment. In addition, the solid line shown with the reference numeral L4 in FIG. 6 represents the resistance force such as the frictional force in the endoscope according to the present embodiment. As shown in FIG. 6, the elastic restoring force L3 is reduced compared with the elastic restoring force (conventional) L1 in the conventional endoscope (see the arrow F1). Similarly, the resistance force L4 is reduced compared with the resistance force (conventional) L2 in the conventional endoscope (see the arrow F2). In the endoscope 1 according to the present embodiment, the amount of force as a result of adding the elastic restoring force L3 and the resistance force L4 is reduced overall.

In addition, FIG. 5 illustrates the state of the bending operation assisting mechanism unit in a case where the rotation operation of the bending operation member is performed and the bending portion is in the middle of being bent. Here, as the example of the middle of the bending operation, the state is shown in FIG. 5 in which as a result of the rotation operation of the bending operation member 22 by the user, the internal gear 63 is rotated from the state shown in FIG. 3 by only the rotation angle of approximately 15 degrees in the clockwise direction, with the rotation center axis C as the center.

As shown in FIG. 5, when the internal gear 63 is rotated in the predetermined direction (clockwise direction in FIG. 5) from the state shown in FIG. 3 by only the rotation angle of 15 degrees, in accordance with the rotation, the pin 66a of the shaft 66 moves in the direction same as the rotation direction of the internal gear 63 (clockwise direction in FIG. 5) along the circular arc having the rotation center axis C as the rotation center. Then, the shaft 66 and the cylinder 65 rotate in the predetermined direction (counterclockwise direction in FIG. 5) by a predetermined amount with the rotation center axis 65a as the pivot. At this time, the shaft 66 is biased in the direction along the arrow X in FIG. 5 by the biasing force of the compression spring 67. In this state, the amount of force X4 with which the compression spring 67 presses the pin 66a of the shaft 66 is resolved into the amount of force X5 that acts in the direction in which the compression spring 67 causes the internal gear 63 to rotate and the amount of force X6 with which the compression spring presses the planetary gear mechanism 64 toward the center thereof. The amount of force X4 with which the biasing force of the compression spring 67 presses the pin 66a is large. In the resolved forces, the proportion of the amount of force X6 for pressing the planetary gear mechanism 64 toward the center thereof is large, and the proportion of the amount of force X5 for rotating the internal gear 63 is small. This shows that the amount of force X5 for pressing the internal gear 63 in the rotation direction, which is generated by the biasing force of the compression spring 67, is set so as not to exceed the resistance forces such as the elastic restoring force, the frictional force, and the like of the bending portion 7 in the middle of the bending operation. That is, when the biasing force of the compression spring 67 acts on the internal gear 63 in the middle of the bending operation, the biasing force is set so as not to actively bend the bending portion 7.

Note that an example is shown in the description above, in which the rotation angle of the bending operation member 22 for bringing the bending portion into the maximum bending state is approximately 180 degrees. However, the rotation angle is not limited to the example. For example, in the endoscope 1, when the distal end constituting portion 6 is bent in the up/down direction, it is preferable to bend the bending portion by around 180 degrees, as described in the example. However, when the distal end constituting portion 6 is bent in the left/right direction, the maximum bending angle may be set to a degree smaller than 180 degrees.

In addition, in the present embodiment, the pin 66a of the shaft 66 is coupled to a part of the internal gear 63. However, the present invention is not limited to the exemplary configuration. For example, the member coupled to a part of the internal gear 63 may be the cylinder 65, and the rotation center axis 65a as the pivot of the cylinder 65 may be coupled to the internal gear. In such a case, the pin 66a of the shaft 66 is configured to be rotatably and pivotally supported with respect to the fixed member such as the main frame 31, for example.

That is, in other words, it has only to be configured that at least one of the distal end of the shaft 66 and the distal end of the cylinder 65 is pivotally supported at the fixed member (for example, immovable portion such as the main frame 31), and at least the other of the distal end of the shaft 66 and the distal end of the cylinder 65 is pivotally supported at the outer diameter portion of the internal gear 63 (output gear).

Furthermore, in the present embodiment, the configuration is exemplified in which the rotation center of the planetary gear mechanism 64, that is, the rotation center axis C of the sun gear 61, and the center axes of the spindle 34, sprocket 33, cylindrical body 36, bending operation member 22, and the like are coaxially arranged. The present invention, however, is not limited to such a configuration. That is, as long as the rotation operation of the bending operation member 22 is transmitted to the planetary gear mechanism 64 through the cylindrical body 36 and the sprocket 33, to surely cause the sun gear 61 to rotate, the rotation center axis of the planetary gear mechanism 64, and the rotation centers of the spindle 34, bending operation member 22, and the like are not necessarily coaxially arranged.

As described above, the first embodiment uses ingenuity to provide the bending operation assisting mechanism unit with an extremely simple configuration in the bending operation mechanism for the endoscope. Therefore, when the bending operation of the bending portion 7 is performed by the rotation operation of the bending operation member 22, various kinds of resistance forces can be reduced particularly when the bending portion is brought into the maximum bending state. As a result, the load to be applied to the user's hand and fingers when the user performs the bending operation can be reduced.

In addition, the present embodiment is capable of contributing to the reduction of the amount of bending operation force in substantially the entire parts which are under the bending operation, without changing the operational feeling at the time of bending operation in the conventional endoscope, for example, the rotation amount of the bending operation member 22.

Furthermore, when the bending portion 7 is returned from the maximum bending state to the linear state, the biasing force of the compression spring 67 is applied in the direction of rotating the internal gear 63. The amount of biasing force acts on the bending portion 7 against the elastic restoring force of the bending portion 7 itself. Therefore, the bending operation assisting mechanism unit 60 prevents the bending portion 7 in the maximum bending state from suddenly returning to the linear state by the elastic force of the bending portion itself.

The bending operation assisting mechanism unit 60 can be implemented with extremely simple configuration, which enables a bending operation mechanism having a mechanism for extremely effectively assisting the bending operation to be provided without much increase in the number of components and without increasing the size of the operation portion. As a result, it is possible to provide an endoscope having extremely excellent sense of use by applying the bending operation mechanism having such a configuration to the endoscope.

Note that it is needless to say that the present invention is not limited to the above-described embodiment, and various modifications and applications are possible in a range without departing from the gist of the invention. Furthermore, the above-described embodiment includes the invention at various stages, and various kinds of inventions can be extracted by appropriately combining the disclosed plurality of constituent elements. For example, even if some constituent elements are deleted from all the constituent elements shown in the embodiment, the configuration from which some constituent elements are deleted can be extracted as the invention if the problem to be solved by the invention can be solved and the effects of the invention can be obtained. Furthermore, constituent elements in different embodiments may be appropriately combined.

The present invention can be applied not only to endoscope control apparatuses in medical fields but also to endoscope control apparatuses in industrial fields.

What is claimed is:

1. A bending operation mechanism for endoscope comprising:
a bending operation member to which a bending wire for driving a bending portion of an endoscope is coupled, the bending operation member being rotationally operated to pull the bending wire in a rotation direction;
a rotator coupled to the bending operation member and configured to be rotationally driven by rotation operation of the bending operation member;
a reduction gear mechanism that reduces rotation of the rotator; and
a biasing mechanism that applies a biasing force in a rotation direction to an output gear in the reduction gear mechanism.

2. The bending operation mechanism for endoscope according to claim 1, wherein
the biasing mechanism includes a proximal end rotatably and pivotally supported at an immovable portion and a distal end rotatably and pivotally supported at an outer diameter portion of the output gear, and
when the bending operation member is in a neutral state in which the bending portion is not bent and in substantially a linear state, two axes of the biasing mechanism and a rotational axis of the output gear are arranged so as to align on substantially a straight line.

3. The bending operation mechanism for endoscope according to claim 2, wherein
the biasing mechanism includes a shaft, a cylinder that pivotally supports the shaft such that the shaft is slidable in an axial direction thereof, and a compression spring bridged between the shaft and cylinder,
at least one of a distal end of the shaft and a distal end of the cylinder is pivotally supported at the immovable portion, and
at least the other of the distal end of the shaft and the distal end of the cylinder is pivotally supported at the outer diameter portion of the output gear.

4. The bending operation mechanism for endoscope according to claim 1, wherein
the reduction gear mechanism is constituted of a planetary gear mechanism,
the planetary gear mechanism including:
a sun gear configured to be rotated integrally with the rotator;
a planetary gear rotatably and pivotally supported at the immovable portion, and configured to be meshed with the sun gear; and
an internal gear provided on an outer circumferential side of the planetary gear, and configured to be meshed with the planetary gear, and
the output gear is the internal gear in the planetary gear mechanism.

5. The bending operation mechanism for endoscope according to claim 4, wherein the sun gear rotates integrally and coaxially with the rotator.

6. A bending operation mechanism for endoscope comprising:
a bending operation member to which a bending wire for driving a bending portion of an endoscope is coupled, the bending operation member being rotationally operated to pull the bending wire in a rotation direction;
a first gear coupled to the bending operation member and rotationally driven by rotation operation of the bending operation member;
a reduction gear mechanism coupled to the first gear and including a second gear to which rotation of the first gear is transmitted after the rotation is reduced; and
a biasing mechanism that applies a biasing force in a rotation direction to the second gear, wherein
the biasing mechanism includes a proximal end rotatably and pivotally supported at an immovable portion and a distal end rotatably and pivotally supported at an outer diameter portion of the second gear, and
when the bending operation member is in a neutral state in which the bending portion is not bent and in substantially a linear state, two axes of the biasing mechanism and a rotational axis of the second gear are arranged so as to align on substantially a straight line.

7. The bending operation mechanism for endoscope according to claim 6, wherein
the first gear is a sun gear in a planetary gear mechanism,
the second gear is an internal gear configured to be meshed with the sun gear through a planetary gear in the planetary gear mechanism, and
the planetary gear is pivotally supported at the immovable portion.
